(19) United States
(12) Patent Application Publication
(10) Pub. No.: US 2019/0096523 A1
(43) Pub. Date: Mar. 28, 2019

(54) HOME CARE OPTIONS COMPLIANCE TABLET FOR POST-ACUTE-CARE PATIENTS

(71) Applicant: Mark Taniguchi, Hercules, CA (US)

(72) Inventor: Mark Taniguchi, Hercules, CA (US)

(21) Appl. No.: 16/203,611

(22) Filed: Nov. 29, 2018

Related U.S. Application Data
(60) Provisional application No. 62/731,884, filed on Sep. 15, 2018.

(51) Int. Cl. G16H 40/20; G16H 80/00; G16H 20/30; G16H 70/20

(52) U.S. Cl. CPC G16H 40/20; G16H 80/00; G06Q 30/0281; G16H 70/20; G16H 20/30

(57) ABSTRACT

A post-acute-care/home-care-options compliance table for post-acute-care patients about to be discharged helps keep hospitals, skilled nursing facilities, residential care facilities for the elderly (RCFE's), and communities in compliance by recording the circumstances, how, and who referred and selected which home care company. Each specific referral source can be recorded as coming from a case manager, social worker, discharge planner, director of assisted living, sales director, executive director, administrator, etc. Archiving such data assists in keeping each referral source in compliance with Medicare, and helps avoid an unintentional violation of the Stark Law. Post-acute-care patients have in their hands an objective and consistent view into all their available home care options, therefore keeping the referral sources themselves clear of many common compliance violations. Legal liabilities involved are reduced, and the post-acute-care patients themselves make their own home care decisions using the solid information placed in their hands with the tablet.

### FIELD OF INVENTION

The present invention relates to helping post-acute-care patients in hospitals choose providers to assist them after discharge. More specifically, a highly modified wireless tablet is placed in the hands of post-acute-care patients by hospital case managers as they are each about to be discharged. The tablets display a level playing field of the home care options available for their specific needs, and can assist as research tools for each patient to find out more before finally deciding. Healthcare Fraud Compliance documentation is easily and automatically recorded.

### BACKGROUND

The referral of post-acute-care patients to post-acute-care and home health agencies and residential care facilities for the elderly has become a big business. The conventional referral practice has been to hand post-acute-care patients a few brochures, typically three, and typically their “preferred” agencies with no other options given to the patient. A compliance violation occurs when referral sources supply only a few home care options, and no others, in which they were receiving some sort of kickback or incentive.

The Federal Government alone pays out billions of dollars every year through Medicare and Medicaid Programs. The home healthcare sector is rife with fraud and abuses that the Federal Government is attempting to control with Legislation like the anti-kickback statutes (AKS), the False Claims Act (FCA), and the Affordable Care Act (ACA).

The anti-kickback statute makes it illegal for providers and physicians to knowingly and willfully accept bribes or other forms of payment in return for generating Medicare, Medicaid or other federal health care program business.

A physician cannot offer anything of value to induce federal health care program business. But, the anti-kickback statute has been revised to allow ever changing exceptions or safe harbors. Legal opinions are needed on how to run these businesses.

Stark II is Phase II of the law that prohibits physician self-referrals. The law applies to any physician who provides care to Medicare, Medicaid or other federal health program recipients. It says that the physician cannot refer post-acute-care patients for certain designated health services to any entity with which the physician has a financial interest. That is, unless one of Stark's exceptions apply.

Stark III is actually Stark II, Phase III of the physician self-referral prohibition. This regulation provides further clarifications and modifications to Stark II, Phase II, especially regarding physicians in group practice and the relationships between physicians and hospitals.

Healthcare providers, like skilled nursing facilities (SNF), are now struggling with compliance issues and looking for new tools that can provide audit trails and assist with compliance reporting.

Big challenges for skilled nursing facilities (SNF's) include: financial pressure, population decline, home healthcare growth, approaching penalties, increased acuity of care, and staffing. SNF's are operating on thin margins in the face of inadequate Medicaid reimbursement while also contending with declining private-pay population numbers. The number of residents for skilled nursing is currently decreasing at a rate of 1% per year. Several new programs allow Medicaid funded services at home and in their communities, thus decreasing the need for inpatient skilled nursing care. As a result, elderly adults are able to go home and stay home. New Penalties for SNF's role in not preventing hospital readmissions are fast approaching. Taking higher acuity post-acute-care patients increases the demands on skilled nursing facilities. It would be easy to turn these post-acute-care patients down, but in order to remain competitive, facilities are accepting these challenging cases. Many facilities still struggle with inadequate nurse staffing levels, which many studies show is a primary cause of hospitalizations.

Because of value-based payment models, hospitals have more at stake and will be holding post-acute-care providers more and more accountable. Post-acute providers have an important role to play, but they must be aggressive. There are several approaches facilities can take to work proactively with hospitals to help improve quality of care and reduce costs. They can start small with just one of these ideas, and then work to build up their efforts. One of the easiest things nursing facilities can do is share quality and patient satisfaction data, as well as materials on services provided. This helps hospitals understand the type of care that residents are receiving. They can also arm hospitals with specific information about how they are working to help reduce readmissions.

Post-acute care networks can spell trouble for SNF's. Hospitals will refer their post-acute-care patients to a small number of SNF's, resulting in high-performing facilities receiving the majority of referrals. Low-performing SNF's may find their occupancy rates dramatically repressed.

The Stark Law, or the Physician Self-Referral Law, is one of many new Federal fraud and abuse laws directed at doctors. Each of the main fraud and abuse Laws is designed to stop a particular kind of well-known fraudulent behavior. The False Claims Act criminalizes filing a fraudulent billing with Medicare and Medicaid. The Stark Law bars doctors from referring post-acute-care patients to entities in which the physician (or an immediate family member) has a financial relationship.

These Laws, in effect, are supposed to neutralize the financial incentives physicians might otherwise realize in referring a patient for treatment after discharge to a home health agency. When a doctor orders equipment or refers a patient to a particular company, it must be because they need the equipment, not because the physician stands to profit. There are far more home health care agencies with qualified equipment and capabilities than is practical for any single health professional to stay abreast of. And so it's natural to stick with those who you're familiar with and have produced good results.

But too much preference and deference can send up a red flag to compliance enforcement that something nefarious is afoot. It would be best if the referrals were spread around the big pool of those available, and then narrowed by informed choices by the post-acute-care patients themselves. Home health agencies need to avoid fraud and the appearance of fraud, but still get in front of as many patient eyeballs as possible.

Both Federal and Massachusetts False Claims Acts prohibit the submission of false claims for third party payment, and provide for the recovery of multiple damages plus significant civil penalties per false claim. Under these laws, private parties can initiate a legal action against an alleged wrongdoer and recover a sizable percentage of any settlement or judgments in the case.

The Stark Law, as it applies to all ten categories of DHS (Stark II), has been in effect since 1995. But not until 2004, have physicians and the health care community had a final set of agency regulations implementing the Law. Many of the changes and additions to the Stark II regulations made by the CMS are intended to ease the burden of compliance with this physician self referral prohibition, and in many cases this has been effective.

But, the Stark Law has become much more complex and harder to follow. The originally intended bright lines are gone. Increasing financial burdens on the federal government to fund the Medicare and Medicaid programs are expected to continue long into the future, the prospect of significant legislative repeal or curtailment of the Stark Law is highly unlikely. The final Stark II regulations, and the expected issuance of final rules on the application of the law to the non-managed care Medicaid Program, scrutiny of physician relationships with medical practices, hospitals, and other providers that render DHS services and items by carriers, enforcement agencies, and private whistleblowers is expected to dramatically increase.

A referral to a physician's wholly owned professional corporation is not a referral if the referring physician personally performs the service. A referral by a physician to his or her own medical practice, regardless of its type of organization, however, is a referral that could violate Stark if some other physician or individual personally performs the referred service. “Referral” is also defined to include services performed or items rendered by the referring physician's employees, independent contractors, or fellow group practice physicians and other professionals. Thus, the Stark Law applies to the ordering of covered services or items to be performed or rendered by the referring physician's employees (including “incident to” services billed as part of the physician's fee), independent contractors, or group practice members. If a physician personally performs the services, there is no referral, regardless of whether the physician bills the programs directly or another entity bills pursuant to an assignment.

Thus, there is no potential Stark violation and no need to try to come within one of the Stark exceptions where there is only a DHS that has been personally performed by the referring physician. However, technical components associated with a physician's personally performed services are referrals to which the Stark Law applies. Thus, if a physician referred and then performed the professional component of an inpatient hospital service payable by Medicare, the physician would still have made a referral to the hospital since it would be separately billing Medicare for the “Part A” or facility or technical component. A physician's prohibited financial relationship with an outside DHS entity (e.g., a home health agency) is not imputed to the physician's entire group practice.

The Stark Law, however, applies to situations in which another person or entity who is controlled or whose referrals are directed by a physician, such as a nurse practitioner, makes the referral. That physician would be viewed as the referring physician. A referral made by someone else in a physician's group, including a non-physician, may be imputed to that physician if he or she directs the referral or otherwise controls the person making the referral. Consultation is defined to mean a professional service furnished to a patient in which the physician's opinion or advice regarding evaluation and/or management of a specific medical problem is requested by another physician; the request and need for consultation are documented in the patient's medical record; and the consulting physician prepares a written report of his or her findings, which is provided to the requesting physician.

All medical practices should incorporate the Stark Law into their on-going internal compliance efforts. Prudent steps in this regard should include identification of all physician financial relationships that come within the scope of Stark, and confirmation of the application of a Stark exception. Paramount among this internal compliance exercise for group practices that render DHS to Medicare and Medicaid post-acute-care patients is a review of each requirement for Stark “group practice” qualification; confirmation that all forty-three referred DHS comes within a group practice exception, and a review and evaluation of current physician compensation methodologies for Stark compliance. Existing documents and contracts should also be periodically reviewed for Stark compliance, as both the group practice and non-group practice exceptions in many instances require the creation and retention of supporting documentation (or written contracts or leases) to satisfy their conditions. Finally, in any instance where there is uncertainty under Stark as to the permissibility of any physician financial relationship or internal medical practice financial arrangement, legal counsel should be consulted, and consideration may need to be given to the potential benefits of seeking a Stark advisory opinion.

What is needed is a personal tool that can be lent briefly to post-acute-care post-acute-care patients to help them make informed and objective choices in providers free of undue and illegal influences.

### SUMMARY

Briefly, designated healthcare services (DHS) referral compliance tablet embodiments of the present invention automate internal compliance efforts, documentation, and reporting for hospitals and physicians under the Stark Law and Anti-Kick-Back Statutes. Group practices that render DHS to Medicare and Medicaid post-acute-care patients are prequalified in a review of each requirement for Stark “group practice” qualification. Confirmation is required that all forty-three referred DHS comes within a group practice exception, and a review and evaluation of current physician compensation methodologies for Stark compliance. Existing documents and contracts are periodically reviewed for Stark compliance, as both the group practice and non-group practice exceptions in many instances require the creation and retention of supporting documentation, or written contracts or leases, to satisfy their conditions. Overall, each physician and group practice should undertake a complete review of all services, procedures, and items they order and/or render to determine if Stark applies to them. Such an analysis is very important—especially when establishing new relationships with other physicians and parties that may be ordering or rendering Stark-covered services or items. Many of the national specialty societies have been quite helpful in identifying which services and procedures within their specialties may be covered under Stark.

### SUMMARY OF THE DRAWINGS

FIG. 1 is a functional block diagram of a home care options compliance tablet and system. A commercial tablet is modified and highly altered to serve post-acute-care patients in hospitals who are about to be discharged and who need to select a home healthcare provider for themselves;

FIG. 2 is a functional block diagram of the electronic processing hardware common to all of the tablets of FIG. 1;

FIG. 3 is a functional block diagram of the commercial tablet software/firmware typical to popular tablets sold by Apple and Samsung;

FIG. 4 is a functional block diagram of the new control program software/firmware that must replace that of FIG. 3 and support a home care options compliance tablet dashboard; and

FIG. 5 is a functional block diagram of a case manager server needed on site with deployed home care options compliance tablet.

### DETAILED DESCRIPTION OF THE EMBODIMENTS

Personal tablets with Wi-Fi and GSM connectivity are proving now they are the enabling hardware that can get Home Care options to post-acute-care patients being discharged from hospitals in a convenient and affordable way. The Stark Law is a “strict liability” statute, so the Government does not need to prove intent to violate the law to find guilt. All that needs to be established is that a Physician, Case Manager and or Social Worker made an inappropriate referral of a patient for a designated home health service where no exception applies.

The Target Buyers of staging home care options in front of post-acute-care patients are the many post-acute home care agencies that need to steer clear of fraud, and yet increase their levels of good clean business in a sea full of pirates and pirate hunters. They can be enrolled as monthly subscribers and maintain the quality and objectivity of referrals through a software-as-a-service (SaaS) installed exclusively on these Tablets.

This is the focus of our intellectual property protection efforts here today. The presentation of objective facts about home care agencies to post-acute-care patients pressed to immediately decide which to choose appears at first to be very straightforward. What then characterizes the Invention is how we limit those choices to the necessary and qualified. And how we enable these post-acute-care patients to communicate, ask questions, and get help. The Invention is further characterized by practical necessities of circulating, distributing, assigning, provisioning, and retrieving Tablets.

There are also security concerns in not allowing too much time to run without gathering a patient's decision, and geo-fencing to warn and retrieve Tablets that wandered outside the facility. So the Solution you invented to deal with a particular aspect of all this is to place in the hands of post-acute-care patients that are about-to-be-discharged from hospitals your single-purposed Tablets that are each are provisioned with a library of home health agencies and their respective services on a leveled playing field. Post-acute-care patients can then browse through the home healthcare services offered by your subscribers.

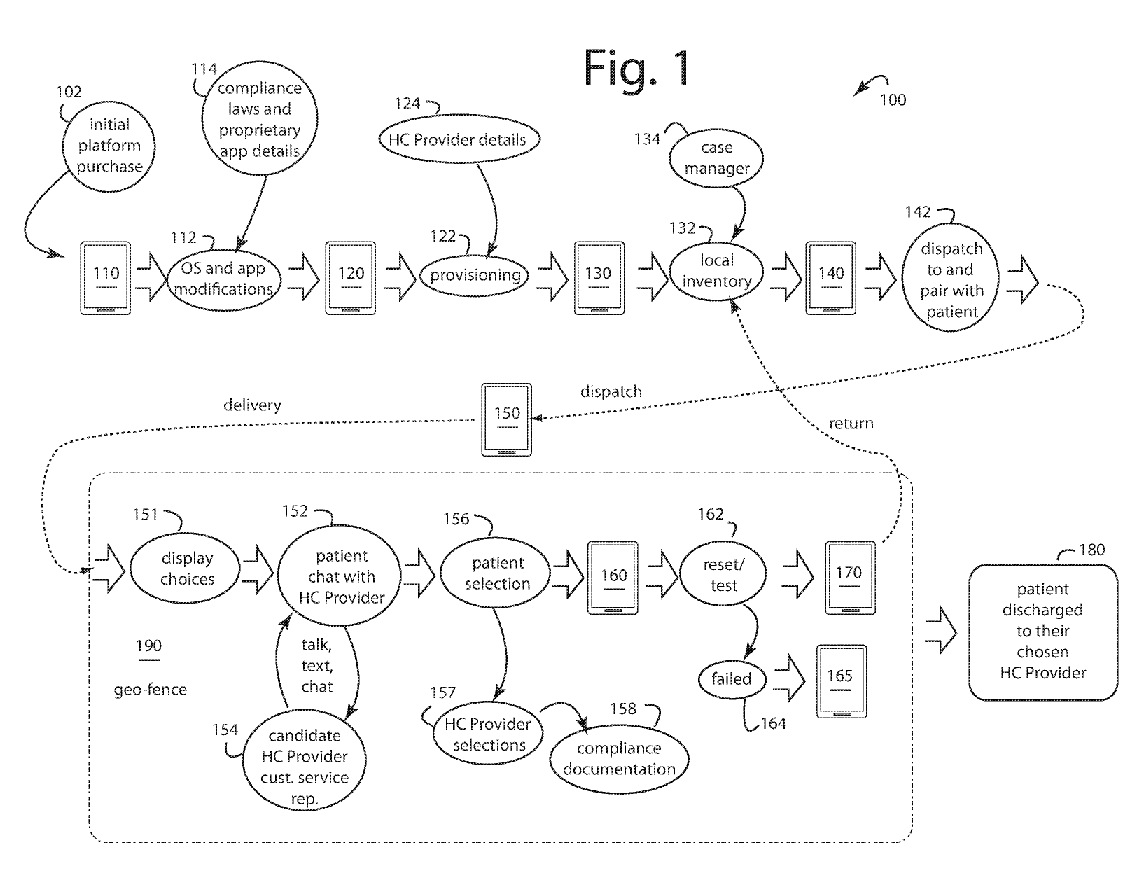

FIG. 1 represents a home care options compliance tablet and system 100. A purchase 102 of a commercially available tablet 110 with a standard operating system is purchased and modified for highly specialized use herein. Acceptable tablets 110 include those with touch-screens, Wi-Fi connectivity, GPS navigation functions, color video display, sound and microphone to support video calls. For example, an Apple iPad or Samsung Tablet.

Tablets 110 cannot be used as-is and must be permanently altered and modified for a couple of reasons. First, the tablet must always boot up into our special application and never present a desktop metaphor or browser. The tablet must run after boot up with our special application as the main control program. Many mobile apps that come standard in tablet 110 are unnecessary and the program and data space they consume must be reserved to our special application.

Second, tablet 110 would be an attractive target for theft if left in its standard commercial configuration. Modified, it has no use except as a patient tool in system 100.

A step 112 permanently modifies and alters tablet 110 with OS and app modification 112. These include special legal constraints and compliance enforcement and reporting tools 114. Our proprietary trade dress, branding, copyright material, specialized library, catalog, and unique look-and-feel are included in 114. The result is a tablet 120.

The key engineering question is how can a designated healthcare services (DHS) referral compliance tablet automate internal compliance efforts, documentation, and reporting for hospitals and physicians under the Stark Law and Anti-Kick-Back Statutes? First, prequalification of referrers to make referrals, and then prequalified to refer to specific DHS providers. Second, who is disqualified, and from what exactly. The Referrals that are made are reportable and require documentation. Statistics help to dismiss favoritism concerns. To show the level playing field.

The common elements necessary for compliance with the Stark Law include Arrangement must be in writing and signed by the parties, Arrangements must have a 1-year duration, Compensation must be set in advance and at fair market value, Compensation must not be determined in a manner that varies with or takes into account the volume or value of referrals or other business generated between the parties, and Arrangements must be commercially reasonable, even in the absence of referrals. A machine cannot make these verifications or judgments, it falls to staff to do that and then input and register the results with system 100. Then, in the compliance documentation that system 100 retains, copies of the signed, written Arrangements are imaged, compensation must be stated, and a qualified opinion by Staff or Counsel that fair market values and commercial reasonability exist, and prohibited terms are not included, like ties of compensation to referral volume. Images of the Arrangements are accessible and viewable on system 100. (But probably not to the post-acute-care patients themselves.)

Ministerial non-compliance includes missing signatures and insufficient writings in the Arrangements.

It follows, system 100 must not allow the display of a DHS Provider or any Referral to them if an accepted Arrangement is not registered and has a staff opinion has not been written as to its acceptability in particular.

The Writing Requirement Standard: “[T]he relevant inquiry is whether the available contemporaneous documents (that is, documents that are contemporaneous with the arrangement) would permit a reasonable person to verify compliance with the applicable exception at the time that a referral is made” (80 FR 71315) Single “formal contract” not required: Collection of documents may satisfy the writing requirement Collection of documents may include “contemporaneous documents evidencing the course of conduct between the parties” (80 FR 71315) Note: Single signed written contract is the best practice and the best way to ensure compliance Relationship of documents in a collection: Documents in the collection must clearly relate to one another Document must clearly evidence one and the same arrangement between the parties The Writing Requirement (cont.) Signature requirement, as applied to a collection of documents: Signature is required on a contemporaneous writing documenting the arrangement Relation to State law State law principles are not dispositive in determining compliance with the writing and signature requirements of the physician self-referral law Parties may look to state law to INFORM the analysis of whether an arrangement is in writing and signed by the parties Clarification of existing policy Guidance regarding the writing requirement is a clarification of existing policy
The 1-year Term Requirement Clarification: Formal “term” provision in a contract not required to satisfy requirement Arrangement with a duration of at least 1 year as a matter of fact satisfies the requirement Written documentation of the term/duration: Contemporaneous documents establishing that the arrangement lasted for at least 1 year, or If the arrangement is terminated during the 1st year, a party must be able to demonstrate that the parties did not enter into a new arrangement for the same space, equipment, or services during the 1st year.

Ensuring Compliance with both the Stark Law and the AKS. The most overarching way organizations can maintain compliance with these laws is by ensuring their compliance programs include seven key elements of an effective compliance program including:

Implementing written policies, procedures and standards of conduct.

Designating a compliance officer and compliance committee.

Conducting effective training and education.

Developing effective lines of communication.

Conducting internal monitoring and auditing.

Enforcing standards through well-publicized disciplinary guidelines.

Responding promptly to detected offenses and undertaking corrective action.

The most notable of these elements as it relates to compliance with the Stark Law and the AKS is implementing written policies, procedures and standards of conduct. By following necessary policies and procedures for these laws, health care organizations can avoid instances of improper referrals and other remuneration fraud or abuse. Compliance Resource Center's Policy Resource Center (PRC) is a beneficial resource for health care organizations that want to make sure they have the right policies and procedures in place to stay compliant with the Stark Law and the AKS.

The “Stark Law” is federal law that prohibits self-referral by physicians, specifically a referral by a physician of a Medicare or Medicaid patient to an entity providing designated health services (“DHS”) that the physician, or an immediate family member, has a financial relationship.

Section 1877 of the Social Security Act, 42 USC 1395 nn, also known as the physician self-referral law and commonly referred to as the “Stark Law” Prohibits a physician from making referrals for certain designated health services (DHS) payable by Medicare to an entity with which they (or an immediate family member) has a financial relationship (ownership, investment, or compensation), unless an exception applies.

The Law prohibits Medicare claims, or billing another individual, entity, or third party payer for those referred services.

Designated health services (DHS) include:
Clinical laboratory services;
Physical therapy services;
Occupational therapy services;
Outpatient speech-language pathology services;
Radiology and certain other imaging services;
Radiation therapy services and supplies;
Durable medical equipment and supplies;
Parenteral and enteral nutrients, equipment, and supplies;
Prosthetics, orthotics, and prosthetic devices and supplies;
Home health services;
Outpatient prescription drugs;
Inpatient and outpatient hospital services.

For physicians in solo or group practices, a threshold consideration under Stark is to first determine whether the practice is rendering and billing for any DHS that is not personally performed by the referring physician. If the solo or group medical practice is doing so, then it is a DHS entity prohibited from billing Medicare and Medicaid for any DHS referred by any physicians with a financial relationship (i.e., all physicians who own the practice and/or receive any form of compensation from the practice), unless those physicians' relationships come within one of the Stark exceptions. Any DHS personally performed or provided by the referring physician is not defined as a “referral” under the Stark Law.

Any DHS performed or provided by any person other than the referring physician, including, but not limited to, the referring physician's employees, independent contractors, or other physicians in the same medical practice, are subject to the Stark prohibitions. In addition, the financial relationship between the referring physician and the practice billing Medicare and Medicaid must fall within one of the enumerated Stark exceptions in order to avoid a Stark violation. This is true even in the case where Medicare permits the services rendered by the other person to be billed under the physician's billing number as “incident to” the referring physician's services. As discussed above, unlike the anti-kickback statute safe harbors, failure to strictly comply with all the requirements of a Stark exception leaves the parties in violation of the law.

For any medical practices subject to Stark, the Physician Services and In-office Ancillary Services exceptions are the most preferable. If all the requirements are met, they cover all of the physicians within a group practice and permit more flexibility in how the practice can permissibly compensate its physicians. These exceptions work differently for solo and group practices. Alternatively, a medical practice with two or more physicians that does provide and bill for Medicare or Medicaid DHS but does not qualify as a group practice under Stark can have distinct compensation arrangements (but not ownership relationships) with individual physicians under the bona fide employment, personal services arrangement, fair-market-value, or academic medical center exceptions. These are the same exceptions that apply to compensation arrangements with hospitals and other DHS entities.

In Rendering DHS Within a Solo Practice Because of the emphasis the Stark Law puts on group practices, a further popular misconception exists that the Stark prohibitions have no potential application to physicians in solo practice. In fact, Stark does apply to a physician in solo practice if they provide and bill for Medicare or Medicaid DHS items or services that are furnished in any manner by other individuals within the practice. For instance, a solo practice physician that employs a technician to render X-rays to Medicare and Medicaid post-acute-care patients is subject to the Stark Law. Most solo practices with a single office location can easily satisfy the in-office ancillary services exception if the physician supervises his staff in conformance with applicable Medicare Part B rules on supervision (see the section entitled “In-Office Ancillary Services Exception”).

The Stark prohibition, which is triggered by any type of compensation arrangement with a DHS entity, is broad enough to encompass common managed care type physician arrangements that include financial incentives to decrease utilization, lower costs, or improve quality. Therefore, whenever a physician or related practice has such a contract with a managed care plan or contracting entity that provides and bills for Medicare or Medicaid DHS, the financial relationship between the physician and the managed care entity is subject to Stark.

COMPARISON OF THE ANTI-KICKBACK STATUTE AND STARK LAW*

| | THE ANTI-KICKBACK | |
|---|---|---|
| | STATUTE | THE STARK LAW |
| | (42 USC § 1320a-7b(b)) | (42 USC § 1395nn) |
| Prohibition | Prohibits offering, paying, | Prohibits a physician from referring Medicare patients |
| | soliciting or receiving anything of | for designated health services to an entity with which |
| | value to induce or reward referrals | the physician (or immediate family member) has a |
| | or generate Federal health care | financial relationship, unless an exception applies |
| | program business | Prohibits the designated health services entity from |
| | | submitting claims to Medicare for those services |
| | | resulting from a prohibited referral |
| Referrals | Referrals from anyone | Referrals from a physician |
| Items/ | Any items or services | Designated health services |
| Services | | |
| Intent | Intent must be proven (knowing | No intent standard for overpayment (strict liability) |
| | and willful) | Intent required for civil monetary penalties for |
| | | knowing violations |
| Penalties | Criminal: | Civil: |
| | Fines up to $25,000 per | Overpayment/refund obligation |
| | violation | False Claims Act liability |
| | Up to a 5 year prison term | Civil monetary penalties and program exclusion for |
| | per violation | knowing violations |
| | Civil/Administrative: | Potential $15,000 CMP for each service |
| | False Claims Act liability | Civil assessment of up to three times the amount |
| | Civil monetary penalties | claimed |
| | and program exclusion | |
| | Potential $50,000 CMP per | |
| | violation | |
| | Civil assessment of up to | |
| | three times amount of | |
| | kickback | |
| Exceptions | Voluntary safe harbors | Mandatory exceptions |
| Federal | All | Medicare/Medicaid |
| Health Care | | |
| Programs | | |

[symbol]

[symbol]

indicates data missing or illegible when filed

It is essential for all physicians and their staffs to fully understand the regulatory interplay and differences between the Stark and anti-kickback laws. The CMS and state-level anti-kickback authorities including the Massachusetts Attorney General's Office have made it clear that satisfaction of the Stark Law does not automatically result in immunity under other fraud and abuse laws, including the federal and state anti-kickback statutes.

These statutes prohibit any individual or entity from knowingly and willfully soliciting, receiving, offering, or paying any form of remuneration (“in cash or in kind”) in order to induce the referral of an individual for the furnishing, or arranging for the furnishing of, any item or service payable under the Medicare or Medicaid programs, or, as is the case in Massachusetts, under any private health plan or insurer. Any party accused of violating the anti-kickback laws can be subject to civil money penalties, criminal prosecution, and/or exclusion from the Medicare and Medicaid programs.

Thus the anti-kickback laws are broader in scope than the Stark physician self-referral prohibition. The federal anti-kickback statute does set forth numerous safe harbors (including those for employment, independent contractors, and group practices) that are similar but not identical to their counterpart Stark exceptions. For example, the anti-kickback safe harbor for independent contractor physicians would only protect contracted services for which the aggregate annual remuneration is set in advance (e.g., $5,000 per month), while the Stark exception counterpart (the personal services arrangement exception) comparatively requires that there be an objectively verifiable compensation formula set in advance, but not the aggregate compensation (e.g., 45% of collections from the physician's personally performed services).

The CMS, however, in two instances did add anti-kickback safe harbors as Stark exceptions (referral services and obstetrical medical malpractice insurance). Any physician financial relationship that is subject to the Stark Law will also be subject to the anti-kickback laws, thus the effort to confirm the regulatory compliance of a physician's financial relationship should not end with Stark.

The relationship must also be considered under the anti-kickback laws. Unlike the Stark Law, which in most situations requires strict adherence to an exception to avoid the prohibition and possible sanctions, the failure to come within an anti-kickback safe harbor does not render a physician's financial relationship per se illegal. At the same time, the federal anti-kickback statute has been interpreted by the federal courts in an extremely broad fashion, so that the government need only prove that a single, and not just the primary, purpose of the remuneration to a physician, even if only offered or solicited but never tendered, is to induce referrals.

The anti-kickback laws require the government to prove that an accused party intended to violate the law. Thus, there is a greater amount of “grey area” under the anti-kickback laws with regard to compliance. Because of this uncertainty, Congress has required the OIG to issue anti-kickback advisory rulings in response to requests from private parties.

Also, all of the Stark exceptions that have an asterisk that do not appear in the original Stark statute require that the financial relationship not violate the federal antikickback laws in order to meet the applicable Stark exception. For example, one of the conditions to meet the Stark fair market value compensation exception, which can protect physician compensation arrangements that do not come within any of the other Stark exceptions, is that the compensation arrangement not violate the federal anti-kickback laws.

In the Stark II regulations, the CMS explained that to fulfill this condition, the parties would have to show that the particular arrangement meets one of the following criteria:
Qualifies as an anti-kickback safe harbor;
Has been specifically approved by the OIG in a favorable anti-kickback advisory opinion;
Does not violate the federal anti-kickback law.

This leaves huge uncertainty in terms of Stark compliance for any such physician financial relationship that does not meet an anti-kickback safe harbor, unless the parties go to the time and expense of seeking an OIG anti-kickback advisory ruling. It is hard to describe these CMS-added Stark exceptions as providing a “bright line” test for physicians and related entities. While compliance with Stark does not automatically cause a physician's relationship to be compliant with the anti-kickback laws, it is likely to bring the relationship closer to being within a safer zone under the anti-kickback statute if it does not fall within any of the anti-kickback safe harbors.

The Stark Law is violated when a referring physician who has a prohibited direct or indirect financial relationship with a DHS entity refers a Medicare- or Medicaid-covered DHS to the DHS entity or when the DHS entity submits claims for payment for such a DHS. Stark provides for two basic types of sanctions: nonpayment/refund of claims imposed on the DHS entity, and civil money penalties for knowing violations imposed on both the DHS entity and referring physician.

Specifically, a violation of the Stark Law can result in the following:
Denial of Medicare or Medicaid payment to the DHS entity for the referred DHS services or items;
Required refund from the DHS entity within 60 days of any amounts billed and collected for the referred DHS services or items;
Imposition of a civil money penalty (CMP) on any referring physician or DHS entity for each bill or claim that the physician or DHS entity knows or should know is for referred services for which payment may not be made (The OIG may impose up to $15,000 in CMPs per wrongful claim and per missed refund, or an assessment of up to triple the amount claimed for each service that was the basis for the CMP.);
Imposition of a CMP of up to $100,000 on any referring physician or DHS entity for each arrangement or scheme the physician/entity knows or should know has a principal purpose of assuring referrals that, if directly made, would violate the Stark Law prohibitions
A five-year exclusion from the Medicare and Medicaid programs for any provider that submits a claim or bill that the provider knows or should know is improper The main sanctions of the Stark Law, denial of payment and required refund, can only be imposed upon the DHS entity and not the referring physician.

Although only DHS entities are liable for this most immediate and easily implemented Stark sanction in the form of payment denials and refunds by the Medicare and Medicaid programs, physicians as owners of medical practices are at great risk under Stark. A Stark violation arises from the failure of a financial relationship between a DHS entity and a referring physician to come within a Stark exception.

Many medical practices are DHS entities and the required refund of any DHS payments made to a medical practice could easily add up to a substantial amount if the Stark violation existed over the course of multiple months or years. For example, a group practice that fails to compensate its physicians consistently with the Stark requirements for group practices and provides in-office radiology services could be required to refund all Medicare and Medicaid payments for the radiology services, and the group practice itself, as well as each referring physician, could be subject to CMPs of up to $15,000 per refunded claim.

Referring physicians as well as DHS entities can be assessed a CMP of up to $15,000 per violation (i.e., a separate CMP per prohibited DHS referral, per prohibited claim for DHS payment, and per missed required refund). Both the referring physician and DHS entity can also be assessed a CMP of up to $100,000 per arrangement that is intended to circumvent the Stark prohibition.

In order to assess CMPs, the government would have to prove that the referring physician or DHS entity had knowledge or should have knowledge of the Stark violation. REPORTING OBLIGATIONS Under the final Stark II regulations, there are no affirmative periodic reporting obligations concerning an entity's ownership and compensation arrangements. Instead, DHS entities must submit certain required information only if they are requested to do so by either the CMS or the OIG. The information that can be required to be disclosed includes: the names and unique physician identification numbers of all physicians who (or whose immediate family members) have an ownership or investment interest in or compensation arrangement with the entity, and with respect to each such physician, the nature of the financial relationship, as evidenced by documentation, routinely maintained by the entity in the course of its business.

DHS entities can also be required to produce documentation supporting compliance with certain Stark exceptions, including group practice eligibility and temporary compliance lapses. If a request for documents is made, the DHS entity must respond within the time period specified in the request, which will be at least 30 days. Failure to meet Stark Law reporting obligations subjects a provider to a civil money penalty of up to $10,000 for each day of noncompliance, and possible Medicare Program exclusion.

Tablet 120 must be provisioned with the latest in approved home healthcare provider details 124. These level the playing field amongst the many qualified home healthcare providers that pass muster legally and professionally. These home healthcare provider details 124 are updated constantly to reflect new information, newly qualified home healthcare providers, and removal of no longer qualified home healthcare providers. The provisioning 122 produces a tablet 130.

Tablets 130 are collected into a local inventory 132 by a case manager 134. Here they sit in secure storage in a hospital ready room near post-acute-care patients who are about to be discharged and who will require on-going out-patient services, equipment, and supplies from the home healthcare providers.

Most such on-going out-patient services, equipment, and supplies from the home healthcare providers will be covered under the post-acute-care patients' healthcare insurance or government programs and entitlements. Such home healthcare industry has been plagued with all manner of fraud, including placing undue influence on post-acute-care patients and facility discharge planners or controlling their choices in engaging on-going out-patient services, equipment, and supplies from the home healthcare providers.

The case manager 134 decides when a particular patient should receive an assigned tablet 140 from the inventory of tablets 130. A step 142 dispatches tablet 140 to the particular patient and engages physical security precautions like traditional asset control and more recent geo-fencing technology.

Post-acute-care patients who will receive tablet 140 will have it paired to them so that only they or a family member (if patient is not capable) can use it. Simple security methods can be included to ensure only the assigned patient is the actual user. The post-acute-care patients receiving tablet 140 will receive the tablet when they must choose a provider, equipment, or supplies within one of five sectors, (1) home care, (2) home health, (3) hospice, (4) placement, and (5) residential care facilities for the elderly (RCFE).

In-home care services are specifically designed to eliminate the need for a senior care facility. With a skilled in-home care services provider post-acute-care patients can continue to enjoy the comforts of home.

Home Health Aide (HHA) services are medically oriented tasks required to maintain the recipient's health or to facilitate treatment of an illness or injury. Services must be ordered by a physician and have professional supervision provided by a Medicare Certified agency.

Hospices are agencies that provide care to terminally ill persons who have a life expectancy of 6-12 months. This care can be provided in the person's home or nursing home, community based residential facility, or another setting.

Individual Placement and Support (IPS), aka supported employment, is a type of employment program that helps individuals with severe and persistent mental illnesses. Research has shown it to be effective for people with many different diagnoses, educational levels, and prior work histories.

Residential Care Facilities for the Elderly (RCFE) serve persons 60 and older. They provide room, board, housekeeping, supervision, and personal care assistance with basic activities like personal hygiene, dressing, eating, and walking. Facilities usually centrally store and distribute medications for residents to self-administer.

This level of care and supervision is for people who are unable to live by themselves but who may not need 24-hour nursing care. They are considered non-medical facilities and are not required to have nurses, certified nursing assistants or doctors on staff. Other terms used to refer to this level of care are assisted living facilities, board and care homes, rest homes and that component of Continuing Care Retirement Communities (CCRCs) that provide personal care and supervision.

Residential Care Facilities for the Elderly or Assisted Living Facilities must meet care and safety standards set by the State and are licensed and inspected, e.g., by the Department of Social Services, Community Care Licensing (CCL).

When a patient has selected a general sector, or when a doctor or the case manager has preselected one of the five sectors, tablet 140 will display several alternative providers on a page of a screen display 151. The display is both comprehensive of all qualified providers and exclusive of providers who are qualified and recognized as such. The patient cannot make a wrong choice or an erroneous choice because under the control program, only those providers in sectors relevant to that ordered by the physician for the particular patient can reach that patient's eyes on the tablet 150.

The providers benefit too from fair rules-of-play and equal opportunities to win business in a compliant setting.

The patient is expected to choose one of these home healthcare options for their ongoing post discharge needs. Some, however will be unable to emotionally or physically. A tablet 150 may help or not help with these issues. Assistant may be required, and tablet 150 documents who assisted with what in particular.

A step 152 allows the patient to click on a home healthcare provider displayed to read/download more information, or to talk, text, or chat 154 with a candidate home healthcare provider customer service representative in real-time.

The patient makes their choice in a step 156, and these are announced, recorded, and notified in a step 157. These selections and the environment they were made in are added to compliance documentation in a step 158. Each patient is free to chose for themselves how to evaluate the candidates and apply whatever criteria they think important.

Once the patient has concluded their selections, the tablet 160 is made inert. That is to prevent unauthorized use. A step 162 tests tablet 160 and resets it to the condition of tablet 130. If tablet 160 fails its tests in a step 164, it's marked as tablet 165 and taken out of service.

Otherwise, it's marked as a tablet 170 and returned to the local inventory 132 and case manager 134.

Then after, the patient is discharged to their chosen home healthcare provider in a step 180.

A geo-fence 190 is setup around the patient recovery room areas in the hospital to notify the case manager 134 of any tablet 150 that have been moved to someplace else. For example, during a theft.

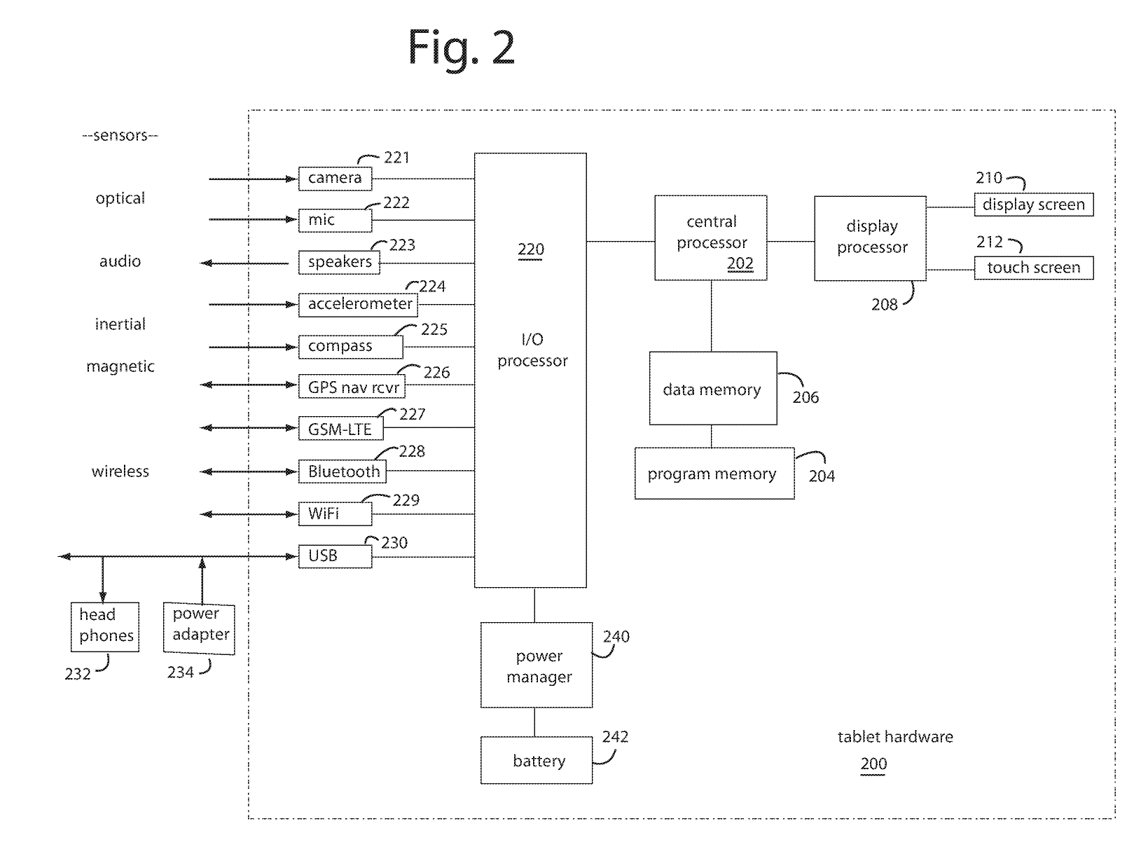

FIG. 2 represents an electronic processing hardware 200 common to tablets 110, 120, 130, 140, 150, 160, 165, and 170 of FIG. 1. Not all of the functionality possible with electronic processing hardware 200 is needed immediately, later, or ever. But the capabilities of electronic processing hardware 200 allow wide latitude in the jobs tablet 150 can perform with changes and upgrades in its programming.

The design, engineering, and manufacture of electronic processing hardware 200 would be a major and expensive undertaking, and certainly not warranted for anything short of mass production in the millions of units. So electronic processing hardware 200 is preferably hijacked from available commercial tablets like the Apple iPad and Samsung Galaxy Note. Their current hardware arrangements are as essentially shown in FIG. 2 as electronic processing hardware 200. As such, electronic processing hardware 200 will be highly familiar in its physical appearance, operation, and needs for care to most users.

A central processor 202 executes a main program and any specialized program applications stored in a program memory 204. A data memory 206 is used to store video, photo, audio, and data files. A display processor 208 offloads the chores involved in maintaining a screen display 210 and user input from a touch screen 212.

An input/output (I/O) processor 220 offloads peripheral processing chores involved with a wide spectrum of sensors 221-229 for optical, audio, inertial, magnetic, and wireless. These perform functions highly familiar to smartphone and tablet users like camera 221, microphone 222, speakers 223, accelerometer (tilt) 224, magnetic compass 225, GPS navigation 226, mobile telephone network (GSM-LTE) 227, Bluetooth connectivity 228, and Wi-Fi 229.

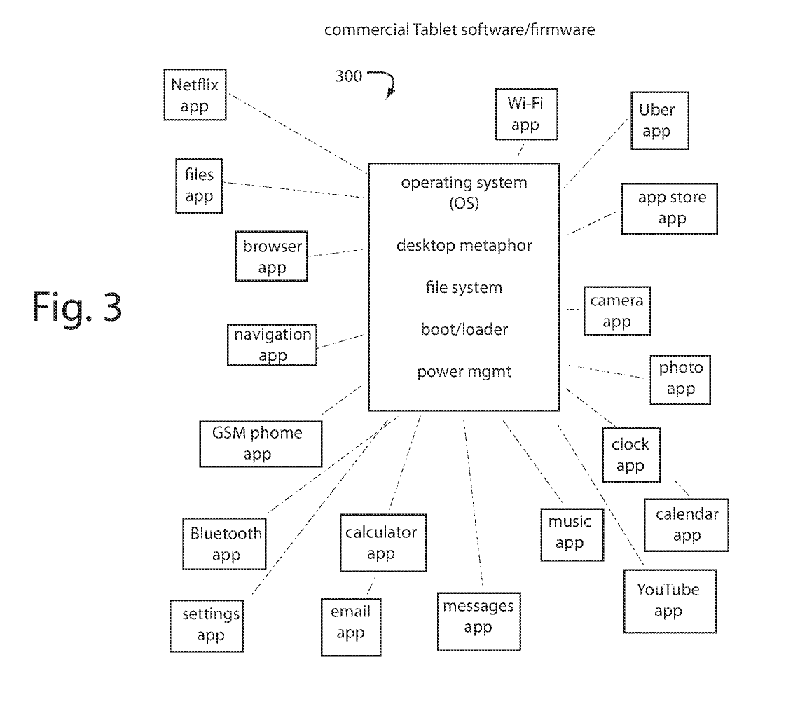

FIG. 3 represents a commercial tablet software/firmware 300 typical of what is usually installed in electronic processing hardware 200 by the likes of Apple and Samsung. Mobile apps typical to these commercial tablets make them fun and entertaining to have and use. Tablet 150 must not be fun and entertaining to the general public because that would make it an attractive target for theft. Many of the capabilities of commercial tablet software/firmware 300 are just inappropriate in a patient/hospital setting.

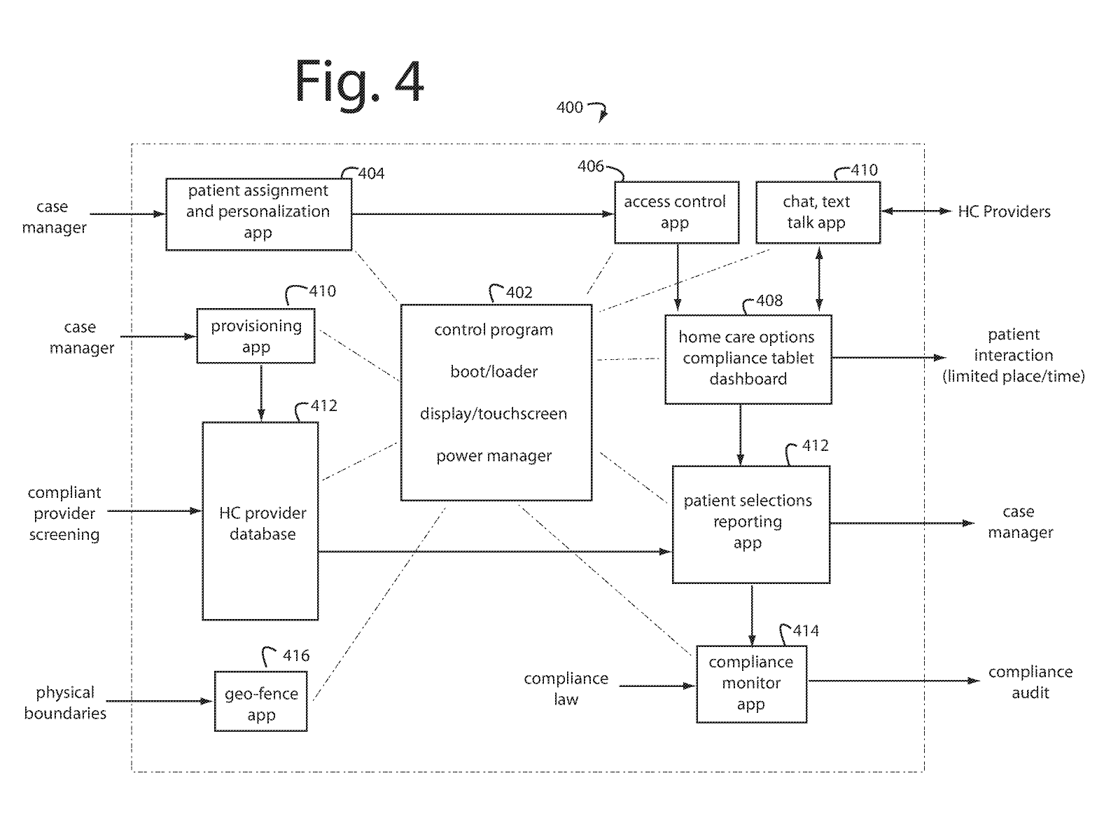

FIG. 4 represents a home care options compliance tablet firmware 400 that completely replaces commercial tablet software/firmware 300 (FIG. 3) which was originally installed by a manufacturer in electronic processing hardware 200 (FIG. 2). A top level control program 402 includes a boot/loader, display and touchscreen controller, and USB/battery power manager. A patient assignment and personalization app 404 receives instructions and data related to the patient being assigned tablet 150. The object of this is to allow gatekeeping by access control app 406. Such gatekeeping limits the person/time/place that a home care options compliance tablet dashboard 408 will be activated and functional.

A provisioning app 410 enables a case manager 134 to input HC provider details 124 into a HC provider database 412 during provisioning 122. Hardware like Bluetooth 228 or Wi-Fi 229 would be a useful means to link in. The HC provider database 412 is filled with compliant providers that have been screened by staff and limited to those relevant to the patient being assigned tablet 150 by case manager 134.

These prescreened and limited HC providers are the only ones that can reach a patient's eyeballs via dashboard 408.

The patient can initiate a chat, text, or talk session with a selected on of the prescreened and limited HC providers in an app 410. Patient selections are reported to the case manager in an app 412. They are also reported for compliance audit with full particulars by a compliance monitor app 414. Current compliance law determines what is required.

A geo-fence app 416 constantly monitors the physical location of tablet 150 using GPS navigation receiver 226 and compares each position fix with physical boundaries 190 supplied by case manager 134. If tablet 150 ranges too far, a notification is sent wirelessly to case manager 134 with details about its illegal location, and dashboard 408 is closed down. Tablet 150 is rendered inert.

The compliance law is ever evolving and open to debate and discussion about what each provision means and how best to effectuate compliance in a patient referral setting. Neither tablet 150 nor its home care options compliance tablet firmware 400 are going to be able to resolve legal subtleties and factual ambiguities. That falls to trained staff who can translate subjective limits imposed by the Law into instructions a machine can understand.

When done right, such tools like tablet 150 become artificial intelligence aids and tools that can help compliance with the anti-fraud legislation and still maintain a profitable business.

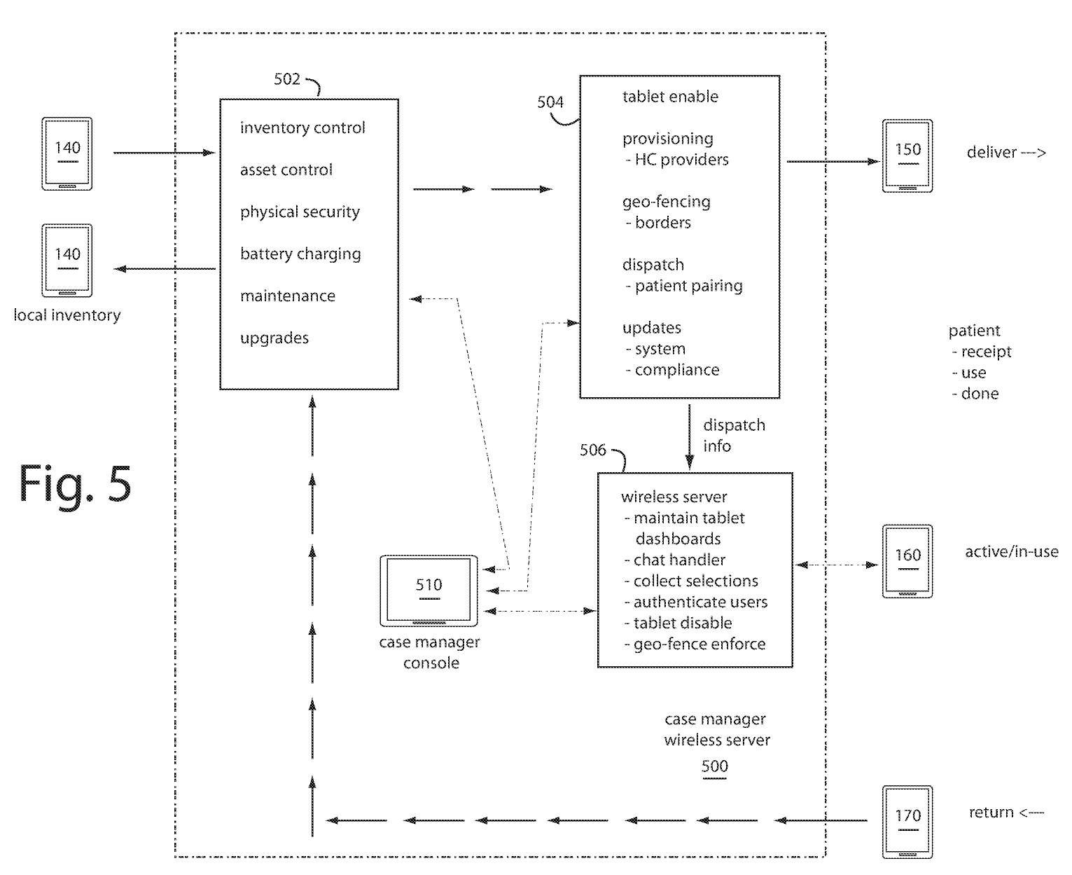

FIG. 5 represents a case manager wireless server 500 that is positioned and operated on the same hospital floor that the post-acute-care patients are using tablets 150. Tablets 140 are received into an inventory control 502 that also includes asset control, physical security for the tablets, battery charging with USB chargers, maintenance, and firmware upgrades or whole tablet generation replacement.

When a need arises, such as a patient has been determined ready for discharge, a process 504 helps the local case manager enable a particular tablet 150 from inventory, updating its location and assignment, provisions the tablet with the correct subset of HC providers that fit the discharge instructions by the responsible physician, establish geo-borders for this particular dispatch, pair the tablet dispatched to the patient being discharged (e.g., passwords, RFID and wrist bracelet barcode readers), and apply any last minute system updates and changes in compliance practice.

The dispatching information from process 504 is forwarded to a network server 506 that establishes and maintains wireless communication with all of dozens of tablets 160 while in active use in its zone of responsibility. (Tablets 150 transform into tablets 160 when an assigned patient logs on and has been authenticated.) Screen displayed dashboards are presented on each tablet 160 and given interactive life by network server 506. If the patient using it wants to engage one of the prescreened HC providers in talk, text, or chat, network server 506 actually handles the connection and records a transcript for later compliance audits.

The selections of HC providers made by the post-acute-care patients at the dashboard are recorded and reported to the case manager. Once selections are complete, the tablet is made inert and non-functional. E.g., to encourage its return to inventory. Geo-fencing is run continually, and any physical excursions of tablet 160 outside the defined borders will sound an alarm and make various notifications. Tablets rendered inert and non-functional are referred to herein as tablets 170.

The case manager and system administrators have access to case manager wireless server 500 via a console 510. Such provides them with operational and administrable control of the whole.

Platform embodiments of the present invention help keep hospitals, skilled nursing facilities, residential care facilities for the elderly (RCFE's), and assisted living communities in compliance by recording who selected which home care company. Each specific referral source can be recorded as a case manager, social worker, discharge planner, director of assisted living, sales director, executive director, administrator, etc.

Keeping such data assists in keeping each referral source in compliance with Medicare, and helps avoid unintentional violation of the Stark Law. For example: referral source-A. (Case Manager), hands a tablet to patient-A. Patient-A has the opportunity to view all home care options on the tablet, therefore keeping the referral source A clear of many common compliance violations.

The conventional practice was that referral source-A would use brochures (generally three brochures) of their “preferred” agencies with no other options given to the patient. This moves referral source-A into compliance violation because referral source-A was giving options of home care options and no others in which they were receiving some sort of kickback.

Our platform also reduces the liability exposure of the referral sources by giving the post-acute-care patients the ability to make their home care decisions. This, opposed to the referral sources making home care decisions for the patient.

A method embodiment of the present invention assists in complying with legal constraints against fraud imposed on acute-care hospitals in the referral of post-acute-care patients to a designated healthcare service (DHS).

Such method autonomously and robotically educates post-acute-care patients as to the existence and qualifications of many locally competing DHS providers. A personal portable electronic device or wireless tablet is placed in their hands by a case worker while the patients are still in their hospital rooms. Such wireless tablets are capable of displaying preapproved, preselected, and preloaded summaries of the DHS providers predetermined by a supervising doctor to be qualified to deliver post-acute-care to this particular post-acute-care patient.

The method further accepts and records any choices of particular DHS providers selected by the acute-care patients. But only after their review of a display and presentation of the existence and qualifications of the plurality of competing DHS providers. The accepting and recording is a conventional peripheral function of the wireless tablet, e.g., by touchscreen or audio input.

The method still further and importantly stores an audit record of the exact things that were displayed and presented to every post-acute-care patient issued the wireless tablet prior to their making choices of particular DHS providers being accepted and recorded, together with the choices themselves.

The displays and presentations that are shown to each post-acute-care patient by the wireless tablets are certified by a staff worker to be uniformly and demonstrably free of favoritism, bias, and preference on the part of the acute-care hospitals amongst the individual competitors in the plurality of competing DHS providers. In other words, a level playing field is established for all the qualified DHS providers, and it is up to the patients to be persuaded which is best for them.

In order to guarantee that it is the assigned patients themselves making informed choices, the accepting and recording of any choices of particular DHS providers selected by the acute-care patient is restricted as to a time period that begins when the acute-care patient is detected by the wireless tablet as having viewed the display and presentation of the existence and qualifications of the plurality of competing DHS providers. And further, the accepting and recording of any choices of particular DHS providers selected by the acute-care patient is also restricted as to a local area that includes a hospital room assigned to the acute-care patient as detected by a geo-fencing and GPS navigation functionality included in the wireless tablet.

Manufacturing from scratch the personal device hardware necessary to support the home healthcare dashboard makes little economic sense. But inventorying Apple iPad or Samsung Galaxy Tab personal devices for use without modification makes the wireless tablet far too attractive targets of theft and competitive pressures. So, the autonomously and robotically educating the post-acute-care patient is such that the wireless tablet placed in their hands is one that was originally manufactured as an equivalent to an Apple iPad or Samsung Galaxy Tab, had its operating system and browser removed and replaced with a home healthcare dashboard and control program that limits the usefulness of the wireless tablet a single application for complying with the then existing legal constraints against fraud imposed on acute-care hospitals in the referral of post-acute-care patients to a designated healthcare service provider.

Although particular embodiments of the present invention have been described and illustrated, such is not intended to limit the invention. Modifications and changes will no doubt become apparent to those skilled in the art, and it is intended that the invention only be limited by the scope of the appended claims.

For example, provider analytics on page views, anonymized data of page views, emotional analysis on each page. If patient is in despair, they would be shown different results. Results can formatted depending on emotion displayed. Augmented reality (AR) capability to quickly view other brochures and contact information for patients (case manager console). Herein, each scan is recorded for analytics. There can be options to do after each scan, adding to case manager profile, view list of previously scanned images/artifacts, e.g., Spotify history of scans and searches.

1. A method of complying with legal constraints against fraud imposed by law on acute-care hospitals in the referral of post-acute-care patients to a designated healthcare service (DHS), comprising:
autonomously and robotically educating a post-acute-care patient as to the existence and qualifications of a plurality of competing DHS providers by placing in their hands while in their hospital rooms a personal portable electronic device or wireless tablet capable of displaying preapproved, preselected, and preloaded summaries of the DHS providers predetermined by a supervising doctor to be qualified to deliver post-acute-care to this particular post-acute-care patient;
accepting and recording any choices of particular DHS providers selected by the acute-care patient after their review of a display and presentation of the existence and qualifications of the plurality of competing DHS providers, wherein the accepting and recording is a peripheral function of the wireless tablet by touchscreen or audio input; and
storing an audit record of the exact things displayed and presented to every post-acute-care patient issued the wireless tablet prior to their choices of particular DHS providers being accepted and recorded, together with the choices themselves;
wherein, the displays and presentations that are shown to each post-acute-care patient by the wireless tablets are certified by a staff worker to be uniformly and demonstrably free of favoritism, bias, and preference on the part of the acute-care hospitals amongst the individual competitors in the plurality of competing DHS providers.

2. The Method of claim 1, wherein:
the accepting and recording of any choices of particular DHS providers selected by the acute-care patient is restricted as to a time period that begins when the acute-care patient is detected by the wireless tablet as having viewed the display and presentation of the existence and qualifications of the plurality of competing DHS providers; and
the accepting and recording of any choices of particular DHS providers selected by the acute-care patient is restricted as to a local area that includes a hospital room assigned to the acute-care patient as detected by a geo-fencing and GPS navigation functionality included in the wireless tablet.

3. The Method of claim 1, wherein:
the autonomously and robotically educating the post-acute-care patient is such that the wireless tablet placed in their hands is one that was originally manufactured as an equivalent to an Apple iPad or Samsung Galaxy Tab, had its operating system and browser removed and replaced with a home healthcare dashboard and control program that limits the usefulness of the wireless tablet a single application for complying with the then existing legal constraints against fraud imposed on acute-care hospitals in the referral of post-acute-care patients to a designated healthcare service provider.

4. The Method of claim 1, wherein:
the storing is such that any significant messages, images, displays, and announcements produced by the wireless tablet, and any of the post-acute-care patient's responses, are recorded and stored in a local wireless server managed by a case worker for later retrieval in support of compliance audits and records.

5. A self-help tablet for the autonomous and robotic referral of post-acute-care patients to designated healthcare services (DHS), comprising:
a commercially marketed tablet with a touchscreen display and Wi-Fi communication capabilities stripped of its original operating system, and thereafter used to host a healthcare dashboard personalized for and operable by a single designated patient;
a navigation receiver included in the tablet and adapted for geo-fencing with time and place boundaries predefined for the operation of the home healthcare dashboard by the single designated patient;
a file included in the tablet that includes service and contacts details of a plurality of DHS providers prequalified to provide healthcare services to the single designated patient, wherein the home healthcare dashboard enables navigation amongst, informational display, and selection by the single designated patient of various ones of the plurality of DHS providers;
wherein, a single post-acute-care patient who has authenticated themselves to the self-help tablet is enabled to choose for themselves a DHS provider for post-acute-care given sterilized, harmonized, and normalized information and summaries directed via the self-help tablet; and
wherein, any significant messages, images, displays, and announcements produced by the self-help tablet, and any of the post-acute-care patient's responses, are recorded and stored for later compliance audits and records.

6. The self-help tablet of claim 5, further comprising:
a wireless server that communicates with and controls the self-help tablet as a compartmentalized member of a group within a facility;
wherein, patient data and access for each single post-acute-care patient is compartmentalized;
wherein, a hospital hosting a group of such self-help tablets is enabled to show or hide DHS providers on each, and to personalize each.

7. An artificial intelligence internal compliance assistant for the referral of designated healthcare services (DHS) to Medicare and Medicaid post-acute-care patients in hospitals immediately before their discharge, comprising:
a personal tablet device limited to Wi-Fi communication with a case manager server on a hospital floor, geo-fenced to operate only on that hospital floor, personalized and authenticated to only one predetermined and designation patient while on that hospital floor, and having an installed control program that does not display a desktop metaphor or browser window, and that further includes a voice, chat, text, and/or data communication ability to contact any displayed DHS provider in real-time;
wherein, a patient assigned and designated to receive and use the personal tablet device is restricted to a limited time frame and restricted area for use that the personal tablet device enforces by not operating outside those restrictions and limitations with its own electronic computations of time and place;
wherein, the DHS providers displayable and selectable by the patient assigned and designated are selected via touch screen and the selections are recorded on a case managers server within wireless communication and copied to a case manager;
wherein, case manager logins are pre-authenticated, such that entering a new session simply requires entering an approved email to be copied;
wherein, the display of DHS information and interfacing options has multi-lingual support;
wherein, all DHS providers that are displayable and selectable have been prequalified as having compliant arrangements with referring physicians in writing with the signed arrangements having digital images of each stored and accessible for screen viewing.

8. The artificial intelligence internal compliance assistant of claim 7, further comprising:
a control program included in the personal tablet device that provides for:
clearing all browsing data from device after each patient completes browsing experience;
emailing to case managers after any browsing experience;
sending of emails to third parties;
case manager collection of referral data that is patient anonymized;
geofencing with info of the floor, assigned patient, room, and alarms when tablet is outside boundary; and
a case manager profile that includes a list of favorited providers for personal recommendations, automatic detection of where tablet was last deployed, without requiring the patient to enter data; and
detection of whether or not a patient is in need of extra help as signposted by emotional analytics, and that then alerts case managers and nurses when patients need suicidal, bipolar, despair, or other immediate specialized attention.

9. The artificial intelligence internal compliance assistant of claim 7, wherein, the geo-fencing includes a GPS navigation computer that calculates the physical position of the tablet, a boundary defined by the case manager that generally includes the hospital floor where the designed patient is assigned, and a notification that alarms and notifies the case manager whenever the tablet is outside the boundary, and that estimates a current position helpful to finding and recovering the tablet.
